(19) United States
(12) Patent Application Publication
(10) Pub. No.: US 2003/0211525 A1
(43) Pub. Date: Nov. 13, 2003

(54) Genes expressed in the cell cycle

(76) Inventors: Michael G Walker, Sunnyvale, CA (US); Kenneth jung, San Francisco, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/362,893

(22) PCT Filed: Aug. 27, 2001
(86) PCT No.: PCT/US01/26682

(51) Int. Cl. C12Q 1/68; C07H 21/04; A61K 48/00

(52) U.S. Cl. 435/6; 514/44; 536/23.1

(57) ABSTRACT

The invention provides cDNAs, their encoded proteins, and antibodies which may be used in methods for diagnosing and treating cell cycle disorders.

### TECHNICAL FIELD

The invention relates to cDNAs identified by their co-expression with known cell cycle genes and to their use in diagnosis, prognosis, treatment, and evaluation of therapies for cell cycle disorders.

### BACKGROUND OF THE INVENTION

Cell division is the fundamental process by which all living things grow, repair, and reproduce. In unicellular organisms, each cell division doubles the number of organisms; and in multicellular species, many rounds of cell division are required to produce a new organism or to replace cells lost by wear and tear or by programmed cell death. Details of the cell division cycle vary, but the basic process consists of three principle events. The first event, interphase, involves preparation for cell division, replication of the DNA, and production of essential proteins. In the second event, mitosis, the nuclear material is divided and separates to opposite sides of the cell. The final event, cytokinesis, is division of the cytoplasm. The sequence and timing of cell cycle events is under the control of cell cycle regulators which control the process by positive or negative mechanisms at various check points.

Cancers and immune conditions, diseases and disorders are associated with the disregulation of normal cell proliferation. In cancer, this disregulation is often attributable to oncogenes, mutant isoformns of normal cellular genes. In some cases, these oncogenes are activated by viruses as a consequence of the integration of a viral genome into the DNA of the host cell. Sometimes, more than one oncogene, capable of maintaining the infected cell in a condition of continuous cell division, is activated. Other oncogenes are abnormally expressed with respect to location or level of expression. This latter category causes cancer by altering transcriptional control of cell proliferation. At least five classes of oncogenes are known; they include cytokines and growth factors; receptors such as erbA, erbB, neu, and ros; intracellular signal transducers such as src, yes, fps, abl, and met; nuclear transcription factors such as fos; cell-cycle control proteins such as RB and p53; and mutated tumor-suppressor genes such as, mdm2, sec, and ras (Bohmann et al. (1987) Science 238:1386-1392; Cohen and Curran (1988) Mol Cell Biol 8:2063-2069; and van Straaten et al. (1983) Proc Natl Acad Sci 80:3183-3187).

For example, in cancer, oncogenes contribute to unrestricted cell proliferation through their involvement in the reception and transduction of growth factor signals and in the modulation of gene expression in response to these signals. Stimulation of a cell by growth factor activates two sets of genes, the early-response genes and the delayed-response genes. Early-response genes include the myc, fos, and jun proto-oncogenes, all of which encode gene regulatory proteins. These regulatory proteins activate the transcription of the delayed-response genes which encode proteins such as the cyclins and cyclin-dependent kinases directly involved in cell cycle progression.

The discovery of cDNAs which coexpress with known cell cycle genes satisfies a need in the art by providing new compositions which are useful in the diagnosis, prognosis, treatment, and evaluation of therapies for cell cycle disorders.

### SUMMARY OF THE INVENTION

The invention provides a composition comprising a plurality of cDNAs having the nucleic acid sequences of SEQ ID NOs: 1-10 or their complements that are coexpressed with one or more known cell cycle genes in a plurality of biological samples. The invention also provides a method of using a composition to screen a plurality of molecules to identify at least one ligand which specifically binds a cDNA of the composition, the method comprising combining the composition with molecules under conditions to allow specific binding; and detecting specific binding, thereby identifying a ligand which specifically binds the cDNA. In one embodiment, the molecules are selected from DNA molecules, RNA molecules, peptide nucleic acids, transcription factors, enhancers, repressors, mimetics, and proteins.

The invention provides a method for using a composition to detect gene expression in a sample containing nucleic acids, the method comprising hybridizing the composition to the nucleic acids under conditions for formation of one or more hybridization complexes; and detecting hybridization complex formation, wherein complex formation indicates gene expression in the sample. In one embodiment, the cDNAs of the composition are attached to a substrate. In another embodiment, complex formation when compared to standards is diagnostic of cell cycle disorders.

The invention provides an isolated cDNA having a nucleic acid sequence selected from SEQ ID NOs: 1, 2, and 4-10 and the complements thereof. In different aspects, each cDNA is used as a diagnostic, as a probe, in an expression vector, and in assessing the prognosis and treatment of a cell cycle disorder. The invention also provides a composition comprising a cDNA and a labeling moiety. The invention further provides a method for using a cDNA to screen a plurality of molecules to identify a ligand which specifically binds the cDNA, the method comprising combining the cDNA with a sample under conditions to allow specific binding; recovering the bound cDNA; and separating the ligand from the bound cDNA, thereby obtaining purified ligand. In one embodiment, the molecules to be screened are selected from DNA molecules, RNA molecules, peptide nucleic acids, transcription factors, enhancers, repressors, mimetics, and proteins.

The invention provides a method for using a cDNA to detect gene expression in a sample containing nucleic acids, the method comprising hybridizing the cDNA to nucleic acids of a sample under conditions for formation of one or more hybridization complexes; and detecting hybridization complex formation, wherein complex formation indicates gene expression in the sample. In one embodiment, the cDNA is attached to a substrate. In another embodiment, gene expression when compared to standards is diagnostic of a cell cycle disorder. The method also provides a vector containing the cDNA, a host cell containing a vector and a method for using a host cell to produce a protein or peptide encoded by the cDNA comprising culturing the host cell under conditions for expression of the protein; and recovering the protein from cell culture.

The invention provides a purified protein encoded by a cDNA of the invention. The invention also provides a method for using the protein or peptide to screen a plurality of molecules to identify and purify a ligand which specifically binds the protein. In one embodiment, the molecules to be screened are selected from DNA molecules, RNA molecules, peptide nucleic acids, proteins, agonists, antagonists, and antibodies.

The invention provides a method of using a protein to prepare and purify antibodies comprising immunizing an animal with the protein or peptide under conditions to elicit an antibody response; isolating animal antibodies; attaching the protein to a substrate; contacting the substrate with isolated antibodies under conditions to allow specific binding to the protein; and dissociating the antibodies from the protein, thereby obtaining purified antibodies. The invention also provides methods for using an antibody which specifically binds the protein to diagnose a cell cycle disorder, the method comprising combining an antibody with a sample under conditions for specific binding, detecting antibody complex formation, comparing antibody complex formation with a standard, thereby diagnosing a cell cycle disorder. The invention further provides a composition comprising a cDNA, a protein or an antibody that specifically binds a protein or peptide and a pharmaceutical carrier for use in treating a cell cycle disorder.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Array” refers to an ordered arrangement of at least two cDNAs or antibodies on a substrate. At least one of the cDNAs or antibodies represents a control or standard, and the other, a cDNA or antibody of diagnostic or therapeutic interest. The arrangement of two to about 40,000 cDNAs or of two to about 40,000 monoclonal or polyclonal antibodies on the substrate assures that the size and signal intensity of each labeled hybridization complex, formed between each cDNA and at least one nucleic acid, or antibody:protein complex, formed between each antibody and at least one protein to which the antibody specifically binds, is individually distinguishable.

“Cell cycle gene” refers to a cDNA which has been previously identified as useful in the diagnosis, prognosis, treatment, and evaluation of therapies associated with unregulated cell cycling. Typically, this means that the known gene is differentially expressed at higher (or lower) levels in tissues from patients with a cell cycle disorder when compared with normal expression in any tissue. The cell cycle genes used in this invention and described in EXAMPLE IV are cdc2, cdc7, cdc23, cyclin B, hBub1, HKSP, hp55cdc, MCAK, mitosin, mki67a, MKLP-1, myb, nlk1, cdc21, PRC1, Aik2, survivin, topoII, and UbcH10.

“Cell cycle disorder” refers to any cancer or immune disorder including, but not limited to, an adenocarcinoma, leukemia, lymphoma, melanoma, myeloma, sarcoma or cancers of the blood, bone, bone marrow, brain, breast, gastrointestinal tract (esophagus, stomach, small intestine or colon), heart, kidney, liver, lung, lymph, muscle, nerve, ovary, pancreas, prostate, skin, spleen, testis, and uterus; asthma, atherosclerosis, Crohn's disease, glomerulonephritis, multiple sclerosis, myasthenia gravis, osteoporosis, rheumatoid arthritis, scleroderma, and systemic lupus erythematosus.

“cDNA” refers to an isolated polynucleotide or any fragment or oligonucleotide thereof. It may of genomic or synthetic origin, double-stranded or single-stranded, and combined with carbohydrate, lipids, protein or other materials to perform a particular activity or form a useful composition.

“Differential expression” refers to an increased or up-regulated or a decreased or down-regulated expression as detected by presence, absence or at least two-fold change in the amount or abundance of a transcribed messenger RNA or translated protein in a sample.

“Isolated or purified” refers to a cDNA or protein that is removed from its natural environment and that is separated from other components with which it is naturally present.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Protein” refers to a polypeptide, or any portion or oligopeptide thereof whether naturally occurring or synthetic.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Similarity” refers to the quantification (usually percentage) of nucleotide or residue matches between at least two sequences aligned using a standard algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a reproducible way to insert gaps in one of the sequences in order to optimize alignment and to achieve a more meaningful comparison between them. Particularly in proteins, similarity is greater than identity in that conservative substitutions (for example, valine for leucine or isoleucine) are counted in calculating the reported percentage. Substitutions which are considered to be conservative are well known in the art.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” is a profile of gene transcription activity in a particular tissue at a particular time.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The present invention utilizes a method for identifying cDNAs or proteins that are associated with a specific disease, regulatory pathway, subcellular compartment, cell type, tissue type, or species. In particular, the method identifies cDNAs useful in diagnosis, prognosis, treatment, and evaluation of therapies for cell cycle disorders.

The method provides for the identification of cDNAs that are expressed in a plurality of libraries. The expression patterns of genes with known function are compared with those of cDNAs with unknown function to determine whether a specified co-expression probability threshold is met. Through this comparison, a subset of the cDNAs having a high co-expression probability with the known genes can be identified.

The cDNAs originate from cDNA libraries derived from a variety of sources including, but not limited to, eukaryotes such as human, mouse, rat, dog, monkey, plant, and yeast; prokaryotes such as bacteria; and viruses. These cDNAs can also be selected from a variety of sequence types including, but not limited to, expressed sequence tags (ESTs), assembled polynucleotides, full length gene coding regions, promoters, introns, enhancers, 5′ untranslated regions, and 3′ untranslated regions. To have statistically significant analytical results, the cDNAs need to be expressed in at least five cDNA libraries.

The cDNA libraries used in the co-expression analysis can be obtained from adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, peritoneum, phagocytes, pituitary, placenta, pleurus, prostate, salivary glands, seminal vesicles, skeleton, spleen, stomach, testis, thymus, tongue, ureter, uterus, and the like. The number of cDNA libraries selected can range from as few as 5 to greater than 10,000. Preferably, the number of the cDNA libraries is greater than 500.

In a preferred embodiment, the cDNAs are assembled from related sequences, such as sequence fragments derived from a single transcript. Assembly of the polynucleotide can be performed using sequences of various types including, but not limited to, ESTs, extension of the EST, shotgun sequences from a cloned insert, or full length cDNAs. In a most preferred embodiment, the cDNAs are derived from human sequences that have been assembled using the algorithm disclosed in U.S. Ser. No. 9,276,534, filed Mar. 25, 1999, incorporated herein by reference.

Experimentally, differential expression of the cDNAs can be evaluated by methods including, but not limited to, differential display by spatial immobilization or by gel electrophoresis, genome mismatch scanning, representational difference analysis, and transcript imaging. Representative transcript images for SEQ ID NO:s 1, 5 and 10 are found in EXAMPLE XV. The transcript images confirm the data produced by the co-expression method disclosed herein. Additionally, differential expression can be assessed by microarray technology. Any of these methods may be used alone or in combination.

Known cell cycle genes can be selected based on function and the use of the genes as diagnostic or prognostic markers or as therapeutic targets for diseases associated with unregulated cell proliferation. Preferably, the known cell cycle genes include cdc2, cdc7, cdc23, cyclin B, hBub1, HKSP, hp55cdc, MCAK, mitosin, mki67a, MKLP-1, myb, nlk1, cdc21, PRC1, Aik2, survivin, topoII, and UbcH10.

The procedure for identifying cDNAs that exhibit a statistically significant co-expression pattern with known cell cycle genes is as follows. First, the presence or absence of a gene sequence in a cDNA library is defined: a gene is present in a cDNA library when at least one cDNA fragment corresponding to that gene is detected in a cDNA sample taken from the library, and a gene is absent from a library when no corresponding cDNA fragment is detected in the sample.

Second, the significance of gene co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 384) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001, more preferably to less than 0.00001.

To determine whether two genes, A and B, have similar co-expression patterns, occurrence data vectors can be generated as illustrated in the table below. The presence of a gene occurring at least once in a library is indicated by a one, and its absence from the library, by a zero.

| | Library 1 | Library 2 | Library 3 | . . . | Library N |
|---|---|---|---|---|---|
| Gene A | 1 | 1 | 0 | . . . | 0 |
| Gene B | 1 | 0 | 1 | . . . | 0 |

For a given pair of genes, the co-occurrence data is summarized in a 2×2 contingency table (below).

| | Gene A Present | Gene A Absent | Total |
|---|---|---|---|
| Gene B Present | 8 | 2 | 10 |
| Gene B Absent | 2 | 18 | 20 |
| Total | 10 | 20 | 30 |

The contingency table shows the co-occurrence data for gene A and gene B in a total of 30 libraries. Both gene A and gene B occur 10 times in the libraries, and the table summarizes and presents: 1) the number of times gene A and B are both present in a library; 2) the number of times gene A and B are both absent in a library; 3) the number of times gene A is present, and gene B is absent; and 4) the number of times gene B is present, and gene A is absent. The upper left entry is the number of times the two genes co-occur in a library, and the middle right entry is the number of times neither gene occurs in a library. The off diagonal entries are the number of times one gene occurs, and the other does not. Both A and B are present eight times and absent 18 times. Gene A is present, and gene B is absent, two times; and gene B is present, and gene A is absent, two times. The probability (“p-value”) that the above association occurs due to chance as calculated using a Fisher exact test is 0.0003. Associations are generally considered significant if a p-value is less than 0.01 (Agresti, supra; Rice, supra).

This method of estimating the probability for co-expression of two genes males several assumptions. The method assumes that the libraries are independent and are identically sampled. However, in practical situations, the selected cDNA libraries are not entirely independent, because more than one library may be obtained from a single subject or tissue. Nor are they entirely identically sampled, because different numbers of cDNAs may be sequenced from each library. The number of cDNAs sequenced typically ranges from 5,000 to 10,000 cDNAs per library. In addition, because a Fisher exact co-expression probability is calculated for each gene versus 37,071 other assembled genes that occur in at least five libraries, a Bonferroni correction for multiple statistical tests is used.

Using the method above, we have identified cDNAs that exhibit strong association, or co-expression, with known genes that are specific to the cell cycle. The results presented in the co-expression table seen in EXAMPLE V are summarized in the table below. Column 1 is the SEQ ID number, column 2, the known cell cycle gene(s) with which the cDNA is most highly co-expressed; column 3, the p-value; and column 4, a cell cyle disorder for which the co-expressed cDNA is a specific diagnostic marker.

| SEQ ID | Cell Cycle Gene | p-value | Cell Cycle Disorder |
|---|---|---|---|
| 1 | topo II | 16 | peritoneal neuroendocrine |
| | | | carcinoid |
| 2 | PRC1 | 12 | colon adenocarcinoma |
| 3 | CDC23 | 12 | lymphoma |
| 4 | topo II, PRC1 | 10 | metastatic melanoma |
| 5 | cyclin B, UbcH10 | 13 | breast cancer |
| 6 | PRC1 | 16 | colon adenocarcinoma |
| 7 | cyclin B | 9.5 | brain cancer |
| 8 | topo II | 13 | testicular adenocarcinoma |
| 9 | topo II | 9 | metastatic melanoma |
| 10 | hp55cdc | 17 | colon adenocarcinoma |

This table shows that the cDNAs claimed herein have a very highly significant co-expression (less than 0.00000001) with known cell cycle genes . Therefore, the cDNAs are useful as surrogate markers in diagnosis, prognosis, and evaluation of therapies for cell cycle disorders and potentially serve as therapeutics for the elimination or control of unregulated cell cycling. Further, the proteins or peptides expressed from the cDNAs are either potential therapeutics or targets for the identification or development of therapeutics. Similarly, antibodies made from or identified using the protein are either potential therapeutics or pharmaceutical carriers.

Therefore, in one embodiment, the present invention encompasses a composition of cDNAs comprising the nucleic acid sequences of SEQ ID NOs: 1-10 or the complements thereof. These ten cDNAs are shown by the method of the present invention to have strong co-expression with known cell cycle genes and with each other. The invention also provides a cDNA, its complement, and a probe comprising the cDNA selected from SEQ ID NOs: 1, 2, and 4-10. Variants typically have at least about 70% nucleic acid sequence identity to at least one of these sequences.

The cDNA or the encoded protein may be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403-410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., p 856-853).

Also encompassed by the invention are polynucleotides that are capable of hybridizing to SEQ ID NOs: 1-10, and fragments thereof under stringent conditions. Stringent conditions can be defined by salt concentration, temperature, and other chemicals and conditions well known in the art. Conditions can be selected, for example, by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5×SSC (sodium saline citrate) with 1% sodium dodecyl sulfate (SDS) at 60° C., which permits complex formation between two nucleic acid sequences that contain some mismatches. Subsequent washes are performed at higher stringency with buffers such as 0.2×SSC with 0.1% SDS at either 45° C. (medium stringency) or 68° C. (high stringency), to maintain hybridization of only those complexes that contain completely complementary sequences. Background signals can be reduced by the use of detergents such as SDS, sarcosyl, or TRIION X-100 (Sigma-Aldrich, St. Louis Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning, A Laboratory Manual, Cold Spring Harbor Press, Plainview N.Y.)

A cDNA can be extended utilizing a partial nucleotide sequence and employing various PCR-based methods known in the art to detect upstream sequences such as promoters and other regulatory elements. (See, e.g., Dieffenbach and Dveksler (1995) PCR Primer a Laboratory Manual, Cold Spring Harbor Press, Plainview N.Y.). Additionally, one may use an XL-PCR kit (Applied Biosystems (ABI), Foster City Calif.), nested primers, and commercially available cDNA libraries (Life Technologies, Rockville Md.) or genomic libraries (Clontech, Palo Alto Calif.) to extend the sequence. For all PCR-based methods, primers may be designed using commercially available software (LASERGENE software, DNASTAR, Madison Wis.) or another program, to be about 15 to 30 nucleotides in length, to have a GC content of about 50%, and to form a hybridization complex at temperatures of about 68° C. to 72° C.

In another aspect of the invention, the cDNA can be cloned into a recombinant vector that directs the expression of the protein, or structural or functional portions thereof, in host cells. Due to the inherent degeneracy of the genetic code, other DNA sequences which encode the same or a functionally equivalent amino acid sequence may be produced and used to express the protein encoded by the cDNA. The nucleotide sequences can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In order to express a biologically active protein, the cDNA or derivatives thereof, may be inserted into an expression vector, i.e., a vector which contains the elements for transcriptional and translational control of the inserted coding sequence in a particular host. These elements include regulatory sequences, such as enhancers, constitutive and inducible promoters, and 5′ and 3′ untranslated regions. Methods which are well known to those skilled in the art may be used to construct such expression vectors. These methods include in vitro recombinant DNA techniques, synthetic techniques, and in vivo genetic recombination (Sambrook, supra; Ausubel, supra).

A variety of expression vector/host cell systems may be utilized to express the cDNA. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the cDNA can be transformed into cell lines using expression vectors which may contain viral origins of replication and/or endogenous expression elements and a selectable or visible marker gene on the same or on a separate vector. The invention is not to be limited by the vector or host cell employed.

In general, host cells that contain the cDNA and that express the protein may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the cDNA may be cultured under conditions for the expression and recovery of the protein from cell culture. The protein produced by a transgenic cell may be secreted or retained intracellularly depending on the sequence and/or the vector used. As will be understood by those of skill in the art, expression vectors containing the cDNA may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein may also be used to specify protein targeting, folding, and/or activity Different host cells which have specific cellular machinery and characteristic mechanisms for post-translational activities (e.g., CHO, HeLa, MDCK, HEK293, and WI38) are available from the ATCC (Manassas Va.) and may be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the cDNAs, wholly or in part, are synthesized using chemical or enzymatic methods well known in the art (Caruthers et al. (1980) Nucl Acids Symp Ser (7) 215-233; Ausubel, supra). For example, peptide synthesis can be performed using various solid-phase techniques (Roberge et al. (1995) Science 269:202-204), and machines such as the ABI 431A peptide synthesizer (ABI) can be used to automate synthesis. If desired, the amino acid sequence may be altered during synthesis and/or combined with sequences from other proteins to produce a variant.

Screening, Diagnostics and Therapeutics

The compositions or cDNAs can be used in diagnosis, prognosis, treatment, and selection and evaluation of therapies for cell cycle disorders including, but not limited to, adenocarcinoma, leukemia, lymphoma, melanoma, myeloma, sarcoma or cancers of the blood, bone, bone marrow, brain, breast, gastrointestinal tract (esophagus, stomach, small intestine or colon), heart, kidney, liver, lung, lymph, muscle, nerve, ovary, pancreas, prostate, skin, spleen, testis, and uterus; asthma, atherosclerosis, Crohn's disease, glomerulonephritis, multiple sclerosis, myasthenia gravis, osteoporosis, rheumatoid arthritis, scleroderma, and systemic lupus erythematosus.

The compositions or cDNAs may be used to screen a plurality of molecules for specific binding affinity. The assay can be used to screen a plurality of DNA molecules, RNA molecules, peptide nucleic acids (PNAs), peptides, ribozymes, antibodies, agonists, antagonists, immunoglobulins, inhibitors, proteins including transcription factors, enhancers, repressors, and drugs and the like which regulate the activity of the polynucleotide in the biological system. The assay involves providing a plurality of molecules, combining the cDNA or a fragment thereof with the plurality of molecules under conditions suitable to allow specific binding, and detecting specific binding to identify at least one molecule which specifically binds the cDNA.

Similarly the proteins or portions thereof may be used to screen libraries of molecules or compounds in any of a variety of screening assays. The portion of a protein employed in such screening may be free in solution, affixed to an abiotic or biotic substrate (e.g. borne on a cell surface), or located intracellularly. Specific binding between the protein and the molecule may be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the cDNAs are used for diagnostic purposes to determine the absence, presence, or altered—increased or decreased compared to a normal standard—expression of the gene. The polynucleotide consists of complementary RNA and DNA molecules, branched nucleic acids, and/or PNAs. In one alternative, the cDNAs are used to detect and quantify gene expression in samples in which expression of the cDNA is correlated with disease. In another alternative, the cDNA can be used to detect genetic polymorphisms associated with a disease. These polymorphisms may be detected in the transcript cDNA.

The specificity of the probe is determined by whether it is made from a unique region, a regulatory region, or from a conserved motif. Both probe specificity and the stringency of diagnostic hybridization or amplification (maximal, high, intermediate, or low) will determine whether the probe identifies only naturally occurring, exactly complementary sequences, allelic variants, or related sequences. Probes designed to detect related sequences should preferably have at least 50% sequence identity to any of the cDNAs.

Methods for producing hybridization probes include the cloning of nucleic acid sequences into vectors for the production of mRNA probes. Such vectors are known in the art, are commercially available, and may be used to synthesize RNA probes in vitro by adding RNA polymerases and labeled nucleotides. Hybridization probes may incorporate nucleotides labeled by a variety of reporter groups including, but not limited to, radionuclides such as ³²p or ³⁵S, enzymatic labels such as alkaline phosphatase coupled to the probe via avidin/biotin coupling systems, fluorescent labels, and the like. The labeled cDNAs may be used in Southern or northern analysis, dot blot, or other membrane-based technologies; in PCR technologies; and in microarrays utilizing samples from subjects to detect altered protein expression.

The cDNAs can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of altered levels of expression in the sample indicates the presence of the disease. Qualitative and quantitative methods for comparing the hybridization complexes formed in subject samples with previously established standards are well known in the art.

Such assays may also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the patient begins to approximate that which is observed in a healthy subject. The results obtained from successive assays may be used to show the efficacy of treatment over a period ranging from several days to many years.

The cDNAs may also be used on a microarray to monitor the expression patterns. The microarray may also be used to identify splice variants, mutations, and polymorphisms. Information derived from analyses of the expression patterns may be used to determine gene function, to understand the genetic basis of a disease, to diagnose a disease, and to develop and monitor the activities of therapeutic agents used to treat a disease. Microarrays may also be used to detect genetic diversity, single nucleotide polymorphisms which may characterize a particular population, at the genome level.

In yet another alternative, cDNAs may be used to generate hybridization probes useful in mapping the naturally occurring genomic sequence. Fluorescent in situ hybridization (FISH) may be correlated with other physical chromosome mapping techniques and genetic map data as described in Heinz-Ulrich et al. (In: Meyers, supra, pp. 965-968).

In another embodiment, antibodies or Fabs comprising an antigen binding site that specifically binds the protein may be used for the diagnosis and prognosis of diseases characterized by the over-or-under expression of the protein. A variety of protocols for measuring protein expression, including ELISAs, RIAs, and FACS, are well known in the art and provide a basis for diagnosing altered or abnormal levels of expression. Standard values for protein expression are established by combining samples taken from healthy subjects, preferably human, with antibody to the protein under conditions for complex formation The amount of complex formation may be quantitated by various methods, preferably by photometric means. Quantities of the protein expressed in disease samples are compared with standard values. Deviation between standard and subject values establishes the parameters for diagnosing or monitoring disease. Alternatively, one may use competitive drug screening assays in which neutralizing antibodies capable of binding specifically with the protein compete with a test compound. Antibodies can be used to detect the presence of any peptide which shares one or more antigenic determinants with the protein. In one aspect, the antibodies can be used for treatment or monitoring therapeutic treatment for cell cycle disorders.

In another aspect, the cDNA, or its complement, may be used therapeutically for the purpose of expressing mRNA and protein, or conversely to block transcription or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art can be used to construct vectors to express nucleic acid sequences or their complements. (See, e.g., Maulik et al. (1997) Molecular Biotechnology, Therapeutic Applications and Strategies, Wiley-Liss, New York N.Y.) Alternatively, the cDNA or its complement, may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of the cDNA by transfection, liposome injections, or polycationic amino polymers may be achieved using methods which are well known in the art. (See, e.g., Goldman et al. (1997) Nature Biotechnology 15:462-466.) Additionally, endogenous gene expression may be inactivated using homologous recombination methods which insert an inactive gene sequence into the coding region or other targeted region of the cDNA. (See, e.g. Thomas et al. (1987) Cell 51: 503-512.)

Vectors containing the cDNA can be transformed into a cell or tissue to express a missing protein or to replace a nonfunctional protein. Similarly a vector constructed to express the complement of the cDNA can be transformed into a cell to downregulate the protein expression. Complementary or antisense sequences may consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, enhancers, repressors, or regulatory molecules. Recent therapeutic advances using triplex DNA have been described in the literature. (See, e.g., Gee et al. In: Huber and Carr (1994) Molecular and Immunologic Approaches, Futura Publishing, Mt. Kisco N.Y., pp. 163-177.)

Ribozymes, enzymatic RNA molecules, may also be used to catalyze the cleavage of mRNA and decrease the levels of particular mRNAs, such as those comprising the cDNAs of the invention. (See, e.g., Rossi (1994) Current Biology 4: 469-471.) Ribozymes may cleave mRNA at specific cleavage sites. Alternatively, ribozymes may cleave mRNAs at locations dictated by flanking regions that form complementary base pairs with the target mRNA. The construction and production of ribozymes is well known in the art and is described in Meyers (supra).

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, may be included.

Further, an antagonist, or an antibody that binds specifically to the protein may be administered to a subject to treat a cell cycle disorder. The antagonist, antibody, or fragment may be used directly to inhibit the activity of the protein or indirectly to deliver a therapeutic agent to cells or tissues which express the protein. The therapeutic agent may be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein may be generated using methods that are well known in the art. Such antibodies may include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein may be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used. (See, e.g., Pound (1998) Immunochemical Protocols, Methods Mol Biol Vol. 80). Alternatively, techniques described for the production of single chain antibodies may be employed. Fabs which contain specific binding sites for the protein may also be generated. Various immunoassays may be used to identify antibodies having the desired specificity. Numerous protocols for competitive binding or immunoradiometric assays using either polyclonal or monoclonal antibodies with established specificities are well known in the art.

Yet further, an agonist of the protein may be administered to a subject to treat or prevent a disease associated with decreased expression, longevity or activity of the protein.

An additional aspect of the invention relates to the administration of a pharmaceutical or sterile composition, in conjunction with a pharmaceutically acceptable carrier, for any of the therapeutic applications discussed above. Such pharmaceutical compositions may consist of the protein or antibodies, mimetics, agonists, antagonists, or inhibitors of the protein. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a subject alone or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

For any compound, the therapeutically effective dose can be estimated initially either in cell culture assays or in animal models such as mice, rats, rabbits, dogs, or pigs. An animal model may also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, and most preferably, humans.

### EXAMPLES

It is to be understood that this invention is not limited to the particular devices, machines, materials and methods described. Although particular embodiments are described, equivalent embodiments may be used to practice the invention. The described embodiments are provided to illustrate the invention and are not intended to limit the scope of the invention which is limited only by the appended claims.

I cDNA Library Construction

The LUNGTUT09 cDNA library was constructed from cancerous lung tissue obtained from a 68-year-old Caucasian male during a segmental lung resection following diagnosis of malignant neoplasm of the upper right lobe of the lung. Pathology of the right upper lobe of the lung indicated an invasive grade 3 squamous cell carcinoma forming an infiltrating mass involving the bronchus and the surrounding parenchyma. Patient history includes previous diagnoses of type II diabetes without complications, thyroid disorder, depressive disorder, hyperlipidemia, ulcer of the esophagus, and atherosclerosis. Family history included alcohol use in the mother and father, atherosclerosis in a sibling and a grandparent and malignant brain neoplasm in the mother.

The frozen tissues were homogenized and lysed in TRIZOL reagent (1 g tissue/10 ml; Life Technologies), using a POLYTRON homogenizer (Brinkmann Instruments, Westbury N.Y.). After a brief incubation on ice, chloroform was added (1:5 v/v), and the lysate was centrifuged. The upper chloroform layer was removed to a fresh tube, and the RNA extracted with isopropanol, resuspended in DEPC-treated water, and treated with DNAse for 25 min at 37C. The RNA was re-extracted once with acid phenol-chloroform, pH 4.7, and precipitated using 0.3M sodium acetate and 2.5 volumes ethanol. The mRNA was isolated with the OLIGOTEX kit (Qiagen, Chatsworth Calif.) and used to construct the cDNA library.

The mRNA was handled according to the recommended protocols in the SUPERSCRIPT plasmid system (Life Technologies). The cDNAs were fractionated on a SEPHAROSE CL4B column (Amersham Pharmacia Biotech (APB), Piscataway N.J.), and those cDNAs exceeding 400 bp were ligated into pINCY plasmid (Incyte Genomnics, Palo Alto Calif.). The plasmid was subsequently transformed into DH5α competent cells (Life Technologies).

II Isolation and Sequencing of cDNA Clones

Plasmid DNA was released from the cells and purified using the REAL PREP 96 plasmid kit (Qiagen). The recommended protocol was employed except for the following changes: 1) the bacteria were cultured in 1 ml of sterile TERRIFIC BROTH (BD Biosciences, San Jose Calif.) with carbenicillin at 25 mg/l and glycerol at 0.4%; 2) the cultures were incubated for 19 hours after the wells were inoculated and then lysed with 0.3 ml of lysis buffer; 3) following isopropanol precipitation, the DNA pellet was resuspended in 0.1 ml of distilled water. After the last step in the protocol, samples were transferred to a 96-well block for storage at 4C.

The cDNAs were prepared using a MICROLAB 2200 system (Hamilton, Reno Nev.) in combination with DNA ENGINE thermal cyclers (MJ Research, Watertown Mass.). The cDNAs were sequenced by the method of Sanger and Coulson (1975; J Mol Biol 94:441f) using ABI PRISM 377 DNA sequencing systems (ABI). Most of the sequences were sequenced using standard ABI protocols and kits (ABI) at solution volumes of 0.25×-1.0×. In the alternative, some of the sequences were sequenced using solutions and dyes from APB.

III Selection, Assembly, and Characterization of Sequences

The sequences used for co-expression analysis were assembled from EST sequences, 5′ and 3′ long read sequences, and full length coding sequences. Selected assembled sequences were expressed in at least three cDNA libraries.

The assembly process is described as follows. EST sequence chromatograms were processed and verified. Quality scores were obtained using PHED (Ewing et al. (1998) Genome Res 8:175-185; Ewing and Green (1998) Genome Res 8:186-194), and edited sequences were loaded into a relational database management system (RDBMS). The sequences were clustered using BLAST with a product score of 50. All clusters of two or more sequences created a bin which represents one transcribed gene.

Assembly of the component sequences within each bin was performed using a modification of Phrap, a publicly available program for assembling DNA fragments (Green, P. University of Washington, Seattle Wash.). Bins that showed 82% identity from a local pair-wise alignment between any of the consensus sequences were merged.

Bins were annotated by screening the consensus sequence in each bin against public databases, such as GBpri and GenPept from NCBI. The annotation process involved a FASTn screen against the GBpri database in GenBank. Those hits with a percent identity of greater than or equal to 75% and an alignment length of greater than or equal to 100 base pairs were recorded as homolog hits. The residual unannotated sequences were screened by FASTx against GenPept. Those hits with an E value of less than or equal to 10⁻⁸ were recorded as homolog hits.

Sequences were then reclustered using BLASTn and Cross-Match, a program for rapid amino acid and nucleic acid sequence comparison and database search (Green, supra), sequentially. Any BLAST alignment between a sequence and a consensus sequence with a score greater than 150 was realigned using cross-match. The sequence was added to the bin whose consensus sequence gave the highest Smith-Waterman score (Smith et al. (1992) Protein Engineering 5:35-51) amongst local alignments with at least 82% identity. Non-matching sequences were moved into new bins, and assembly processes were repeated.

IV Description of the Known Cell Cycle Genes

Genes known to be involved in disease processes involving the cell cycle were selected to identify cDNAs. The known genes and a brief description of their functions are found below.

| Gene ID | Name | Description |
|---|---|---|
| 995529 | CDC2 | CDC2, cell division cycle protein 2 (or cyclin B1) is a mitotic kinase |
| | | which triggers entry into mitosis. CDC2 binds chromatin prior to S- |
| | | phase, and is displaced during DNA replication. (Krude et al (1996) |
| | | J Cell Sci 109:309-318; De Souza et al (2000) Exp Cell Res 257:11- |
| | | 21) |
| 336106 | CDC7 | CDC7, cell division cycle protein 7 is a kinase conserved in |
| | | eukaryotes from yeast to humans. It is essential for initiation of |
| | | DNA replication and entry into S-phase. (Donaldson et al. (1998) |
| | | Genes Dev 12:491-501; Jiang et al. (1999) Embo J 18:5703-5713; |
| | | and Masai et al. (1999) Front Biosci 4:834-840) |
| 256671 | CDC23 | CDC23, cell division cycle protein 23, is a component of the |
| | | anaphase-promoting complex that regulates mitosis by catalyzing the |
| | | formation of cyclin B-ubiquitin conjugates, targeting cyclin B for |
| | | degradation. (Prinz (1998) Curr Biol 8:750-760; Zhao et al. (1998) |
| | | Genomics 53:184-90; and Hershko (1999) Philos Trans R Soc Lond |
| | | B Biol Sci 354:1571-1576) |
| 286623 | Cyclin B | Cyclin B is a subunit of cyclin-dependent kinase (cdk) 1. |
| | | Degradation of cyclin B by the anaphase-promoting complex is |
| | | required for inactivation of the kinase and exit from mitosis. CDKs |
| | | are regulators of cell cycle progression and alterations and |
| | | deregulation of CDK activity are characteristic of neoplasia. CDK |
| | | inhibitors and modulators alter cell cycle and induce apoptosis and |
| | | tumor regression. (Hajduch et al. (1999) Adv Exp Med Biol |
| | | 457:341-53; Hershko, supra; and Sausville (1999) Pharmacol Ther |
| | | 82:285-92) |
| 392739 | hBub1 | hBub1, a mitotic checkpoint kinase, is a kinetochore protein that |
| | | monitors chromosome attachment to the spindle in mitotic cells and |
| | | controls exit from mitosis and chromosome segregation. The mitotic |
| | | checkpoint ensures proper chromosome segregation by delaying |
| | | anaphase until chromosomes are aligned on the spindle. Following |
| | | spindle damage, cells exit mitosis and undergo apoptosis. hBub1 is |
| | | required for the checkpoint response to spindle damage; mutations in |
| | | hBub1 disrupt the mitotic checkpoint allowing cells to escape |
| | | apoptosis and continue cell cycle progression, despite spindle |
| | | damage, potentially leading to aneuploidy and contributing to |
| | | neoplasia. (Taylor and McKeon (1997) Cell 89:727-735; Cahill |
| | | (1998) Nature 392:300-303; Ouyang et al. (1998) Cell Growth Differ |
| | | 9:877-885; Imai et al. (1999) Jpn J Cancer Res 90:837-840; Seeley et |
| | | al. (1999) Biochem Biophys Res Commun 257:589-595; and Myrie |
| | | et al. (2000) Cancer Lett 152:193-99. |
| 337334 | hKSP | hKSP, kinesin-like spindle protein (HsEg5), is a spindle-associated |
| | | protein found with centrosomal microtubles during prophase and |
| | | prometaphase centrosome separation, and associated with post- |
| | | mitotic centrosome movement. (Whitehead et al. (1996) Cell Motil |
| | | Cytoskeleton 35:298-308) |
| 201204 | hp55cdc | hp55cdc is a kinetochore and spindle microtuble-associated protein |
| | | that mediates association of the spindle checkpoint protein Mad2 |
| | | with the cyclosome/anaphase promoting complex and is essential for |
| | | cell division. Over expression of p55dcd induces apoptosis. hp55cdc |
| | | is also associated with the mitotic spindle protein kinase Aik. |
| | | (Weinstein et al. (1994) Mol Cell Biol14:3350-3363; Kao et al. |
| | | (1996) Oncogene 13:1221-1229; Kallio et al. (1998) J Cell Biol 141: |
| | | 1393-1406; Kramer et al. (1998) Curr Biol 8:1207-1210; Farruggio |
| | | et al. (1999) Proc Natl Acad Sci 96:7306-7311; and Saffery et al. |
| | | (2000) Hum Mol Genet 9:175-85) |
| 331025 | MCAK | MCAK, mitotic centromere-associated kinesin, is a microtubule |
| | | motor protein recruited to the centromere at prophase that |
| | | participates in anaphase chromosome segregation. (Kim et al. (1997) |
| | | Biochim Biophys Acta 1359:181-186; Maney et al. (2000) Int Rev |
| | | Cytol 194:67-131; Maney et al. (1998) J Cell Biol 142:787-801; |
| | | Wordeman et al. (1999) Cell Biol Int 23:275-86; and Saffery, supra) |
| 26662 | mitosin | Mitosin (CENP-F kinetochore protein) is a nuclear protein that |
| | | associates with centromeres and spindle poles during M phase. |
| | | Overexpression of N-terminally truncated mitosin blocks cell cycle |
| | | progression. Mitosin is correlated with clinical outcome in node- |
| | | negative breast cancer. (Clark et al. (1997) Cancer Res 57:5505-08; |
| | | Zhu (1999) Mol Cell Biol 19: 1016-1024; and Zhu et al. (1997) J Cell |
| | | Biochem 66:441-449) |
| 412661 | mki67a | mki67a (MIB-1) is a definitive cell proliferation marker. It is widely |
| | | used in pathology to measure the growth fraction of cells in human |
| | | tumors. (Schluter et al. (1993) J Cell Biol 123:513-522; Duchrow et |
| | | al. (1995) Arch Immnunol Ther Exp 43:117-121; Dalquen et al. |
| | | (1997) Acta Cytol 41:229-237; and Scholzen and Gerdes (2000) J |
| | | Cell Physiol 182:311-322) |
| 319885 | MKLP-1 | MKLP1, mitotic kinesin-like protein 1, is a spindle-associated |
| | | protein required for mitotic progression. (Nislow et al. (1992) |
| | | Nature 359:543-7; Sharp et al. (1997) J Cell Biol 138:833-843; |
| | | Kobayashi et al. (1998) J Cell Biol 143:1961-70) |
| 977509 | myb | B-myb is a member of the myb family of cell-cycle regulated |
| | | transcription factors, expressed in G1 and S phase. Activity of b- |
| | | myb is stimulated by cyclin A/cdk2-dependent phosphorylation. |
| | | (Robinson et al. (1996) Oncogene 12:1855-64; Saville and Watson |
| | | (1998) Adv Cancer Res 72:109-40; Saville and Watson (1998) |
| | | Oncogene 17:2679-2689; and Horstmann et al. (2000) Oncogene |
| | | 19:298-306) |
| 336560 | NLK1 | NLK1, NIMA-like protein kinase 1, is a human mitotic kinase, |
| | | similar to the NIMA cell-cycle regulatory protein kinase in |
| | | Aspergillus that is essential for entry into and progression through |
| | | mitosis. (Lu and Hunter (1995) Cell 81:413-424; Lu and Hunter |
| | | (1995) Prog Cell Cycle Res 1:187-205; and Shen et al. (1997) Proc |
| | | Natl Acad Sci 94:13618-13623) |
| 347876 | P1-CDC21 | P1-CDC21 is a member of the family of minichromosome |
| | | maintenance proteins essential for DNA replication. (Hu et al. |
| | | (1993) Nucleic Acids Res 21:5289-5293; Ishimi et al. (1996) J Biol |
| | | Chem 271:24115-24122) |
| 411205 | PRC1 | PRC1, protein regulating cytokinesis 1, is a human mitotic-spindle |
| | | associated CDK substrate protein required for cytokinesis. (Jiang et |
| | | al. (1998) Mol Cell 2:877-885) |
| 348211 | Aik2 | The protein kinase Aik2/Aurora2 is localized to the mitotic spindle |
| | | poles, involved in regulating chromosome segregation and |
| | | maintaining genomic stability, and associated with p55cdc/cdc20. |
| | | (Kimura et al. (1999) J Biol Chem 274:7334-40 Kimura et al. |
| | | (1998) Cytogenet Cell Genet 82:147-52; and Farruggio, supra) |
| 251651 | survivin | Survivin is an apoptosis inhibitor expressed in the G2/M phase |
| | | of the cell cycle. At the beginning of mitosis it associates with |
| | | microtubules of the mitotic spindle. It inhibits apoptosis allowing |
| | | cancer cells to survive. (Li et al. (1998) Nature 396:580-584; |
| | | Verdecia et al. (2000) Nat Struct Biol 7:602-608) |
| 232888 | topo II | Topoisomerase II is required for chromosome condensation and |
| | | segregation during DNA replication. Its expression is cell cycle |
| | | dependent; both protein level and catalytic activity peeks in G2/M. |
| | | As part of the regulatory checkpoint at the entry and progression of |
| | | mitosis; it regulates apoptosis. Topoisomerase poisons induce |
| | | carcinogenic chromosomal alterations. (Holm et al. (1989) Mol Cell |
| | | Biol 9:159-168; Kaufmann (1998) Proc Soc Exp Biol Med 217:327- |
| | | 334; Sumner (1995) Exp Cell Res 217:440-447; Anderson and |
| | | Roberge (1996) Cell Growth Differ 7:83-90; Larsen et al. (1996) |
| | | Prog Cell Cycle Res 2:229-239; and Cimini et al. (1997) Cytogenet |
| | | Cell Genet 76:61-67) |
| 235191 | UbcH10 | Cyclin-selective ubiquitin carrier protein (UbcH10/E2-C) catalyzes |
| | | the ubiquitin-mediated proteolysis of mitotic cyclins and is required |
| | | for cells to complete mitosis and enter anaphase of the next cell |
| | | cycle. Mutant UbcH10 inhibits the destruction of cyclins, arrests |
| | | cells in M phase, and inhibits the onset of anaphase. (Townsley et |
| | | al. (1997) Proc Natl Acad Sci 94:2362-2367; Bastians et al. (1999) |
| | | Mol Biol Cell 10:3927-3941) |

V Co-expression Analyses of Known Cell Cycle Genes

Using the LIFESEQ GOLD database (Dec99, Incyte Genomics), we have identified ten cDNAs that show strong association with known cell cycle genes. Initially, degree of association was measured by probability values using a cutoff p-value less than 0.00001. This was followed by annotation and literature searches to insure that the genes that passed the probability test had strong association with known cell cycle genes. The process was reiterated so that an initial selection of 37,071 genes were reduced to the final ten cDNAs claimed herein. The entries in the table below are the negative log of the p-value (−log p) for the co-expression of the two genes. The cDNAs are identified by their LIFESEQ GOLD ID numbers, and the known genes, by their abbreviations as shown above and the number assigned in column 1 which is also used in row 1. The single highest p-values between each of the known genes have been marked in bold. The single highest p-values between at least one known gene and each cDNA is summarized in THE INVENTION section.

| Name/ | | | | | | | | | | | | | | | | | | | |
|---|---|---|---|---|---|---|---|---|---|---|---|---|---|---|---|---|---|---|---|
| Number | 1 | 2 | 3 | 4 | 5 | 6 | 7 | 8 | 9 | 10 | 11 | 12 | 13 | 14 | 15 | 16 | 17 | 18 | 19 |
| 1 CDC2 | NA | | | | | | | | | | | | | | | | | | |
| 2 CDC23 | 13 | NA | | | | | | | | | | | | | | | | | |
| 3 CDC7 | 3.4 | 5.5 | NA | | | | | | | | | | | | | | | | |
| 4 Cyclin B | 12 | 6.6 | 10 | NA | | | | | | | | | | | | | | | |
| 5 hBub1 | 7.8 | 7.7 | 0 | 5.2 | NA | | | | | | | | | | | | | | |
| 6 hKSP | 5.8 | 6 | 5.2 | 10 | 4.9 | NA | | | | | | | | | | | | | |
| 7 hp55cdc | 5.7 | 4.8 | 5.8 | 12 | 5.5 | 7.2 | NA | | | | | | | | | | | | |
| 8 MCAK | 4.8 | 6.9 | 9.2 | 14 | 6.1 | 8 | 11 | NA | | | | | | | | | | | |
| 9 mitosin | 9.8 | 4.6 | 6.6 | 14 | 12 | 7 | 13 | 12 | NA | | | | | | | | | | |
| 10 mki67a | 12 | 5.5 | 8.3 | 6.7 | 6.5 | 5.8 | 7.8 | 11 | 13 | NA | | | | | | | | | |
| 11 MKLP-1 | 6.9 | 5.1 | 4.5 | 3.8 | 6.9 | 5.3 | 8.8 | 3.9 | 7.1 | 7.2 | NA | | | | | | | | |
| 12 myb | 0 | 5.5 | 7.9 | 13 | 5.9 | 5.7 | 19 | 18 | 13 | 20 | 6.8 | NA | | | | | | | |
| 13 NLK1 | 0 | 4.3 | 0 | 0 | 10 | 3.5 | 4.2 | 4.8 | 8.3 | 3.9 | 3.9 | 0 | NA | | | | | | |
| 14 P1- | 11 | 7.4 | 7 | 10 | 6.4 | 5.6 | 14 | 9.2 | 12 | 21 | 5.5 | 10 | 5.6 | NA | | | | | |
| CDC21 | | | | | | | | | | | | | | | | | | | |
| 15 PRC1 | 7.2 | 5.4 | 5.9 | 15 | 9.7 | 12 | 16 | 12 | 13 | 13 | 4.6 | 8.5 | 7.4 | 15 | NA | | | | |
| 16 prkAik2 | 6.6 | 8.4 | 3.2 | 11 | 5 | 6.8 | 9.3 | 6.7 | 7.6 | 11 | 5 | 8.1 | 3.8 | 7.2 | 10 | NA | | | |
| 17 survivin | 11 | 6.5 | 9.2 | 11 | 9.3 | 9.2 | 18 | 11 | 18 | 9 | 10 | 11 | 6.1 | 11 | 7.9 | 9.8 | NA | | |
| 18 topo II | 23 | 11 | 13 | 17 | 11 | 15 | 19 | 14 | 24 | 15 | 6.1 | 18 | 8.2 | 19 | 18 | 11 | 23 | NA | |
| 19 UbcH10 | 6.1 | 5.8 | 4 | 12 | 7.5 | 12 | 15 | 8.7 | 12 | 12 | 5.6 | 16 | 6.2 | 9.4 | 8 | 15 | 16 | 11 | NA |
| 40371 | 7.9 | 3.9 | 5.8 | 4.6 | 9.4 | 6.6 | 5.5 | 7.6 | 7.6 | 8.8 | 7.5 | 7 | 9.1 | 8.5 | 8.7 | 6.4 | 5.8 | 16 | 10 |
| 200394 | 7.1 | 5.2 | 4.9 | 7.6 | 6.5 | 7.5 | 8.9 | 6.8 | 4.5 | 7.3 | 9 | 4.3 | 4.2 | 9.5 | 12 | 6.6 | 5.2 | 11 | 7.1 |
| 201989 | 5.9 | 12 | 4.5 | 10 | 4.5 | 5.4 | 8.3 | 9.5 | 6.7 | 9.6 | 3.2 | 9.7 | 0 | 11 | 8.5 | 11 | 8 | 11 | 11 |
| 211475 | 9.2 | 6.6 | 4.8 | 7.9 | 4.5 | 5.9 | 8.2 | 6 | 5.5 | 6.7 | 4.3 | 4.9 | 0 | 9.8 | 10 | 7.6 | 5.4 | 10 | 6.2 |
| 225657 | 4.7 | 5 | 5 | 13 | 4.5 | 4.5 | 7.9 | 9.8 | 11 | 8.3 | 3.7 | 7.2 | 3.7 | 6.4 | 5.5 | 11 | 8.4 | 6.7 | 13 |
| 350770 | 6.5 | 6.5 | 9.9 | 7.2 | 5.8 | 5.9 | 12 | 11 | 9.1 | 12 | 0 | 11 | 3.7 | 15 | 16 | 8 | 6.5 | 14 | 11 |
| 407614 | 6.6 | 0 | 5.5 | 9.5 | 0 | 3.6 | 7.9 | 7.6 | 4.9 | 4.8 | 3.8 | 6.5 | 0 | 3.5 | 5.9 | 4.6 | 8.2 | 9.4 | 5.6 |
| 475113 | 9 | 10 | 4.2 | 6.7 | 7.6 | 8.6 | 9.1 | 5.1 | 9.3 | 9.4 | 5.7 | 10 | 4.5 | 9 | 10 | 8.4 | 9 | 13 | 12 |
| 898622 | 0 | 0 | 0 | 5.2 | 5.6 | 7.9 | 3 | 0 | 4.9 | 0 | 0 | 0 | 3.3 | 5.2 | 4.2 | 4 | 6.6 | 9 | 3.2 |
| 978267 | 0 | 3.4 | 10 | 7.8 | 3.8 | 5 | 17 | 9.5 | 8 | 4.1 | 4.2 | 0 | 4.2 | 7.8 | 8.2 | 5.6 | 9.4 | 15 | 5.6 |

VI Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10-25 for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda Md.; http://www.ncbi.nlm.nih.gov/gorf/bl2.html), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database. Component sequences from cDNA, extension, full length, and shotgun sequencing projects were subjected to PHED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of <1×10⁻⁸. The templates were also subjected to frameshift FASTx against GENPEPT, and homolog match was defined as having an E-value of <1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the MMER software package (Washington University School of Medicine, St. Louis Mo.; http://pfam.wustl.edu/).

The cDNA was further analyzed using MACDNASIS PRO software (Hitachi Software Engineering), and LASERGENTE software (DNASTAlR) and queried against public databases such as the GenBank rodent, mammalian, vertebrate, prokaryote, and eukaryote databases, SwissProt, BLOCKS, PRINTS, PFAM, and Prosite.

VII Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding tumor antigen that have been mapped result in the assignment of all related regulatory and coding sequences mapping to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VIII Hybridization Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0. 1% SDS and acetone, etching in 4% hydrofluoric acid (VWR Scientific Products, West Chester Pa.), coating with 0.05% aminopropyl silane (Sigma-Aldrich) in 95% ethanol, and curing in a 110C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C.; then the arrays are washed in 0.2% SDS and rinsed in distilled water as before .

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100C. for five min, and briefly centrifuging. The denatured cDNA is then added to a REDIPRRIME tube (APB), gently mixed until blue color is evenly distributed, and briefly centrifuged. Five μl of [³²P]dCTP is added to the tube, and the contents are incubated at 37C. for 10 min. The labeling reaction is stopped by adding 5 μl of 0.2M EDTA, and probe is purified from unincorporated nucleotides using a PROBEQUANT G-50 microcolumn (APB). The purified probe is heated to 100C. for five min, snap cooled for two min on ice, and used in membrane-based hybridizations as described below.

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNase inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C. for two hr. The reaction mixture is then incubated for 20 min at 85C., and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C. for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C. for two hr. The probe, diluted in 15 ml fresh hybridization solution, is then added to the membrane. The membrane is hybridized with the probe at 55C. for 16 hr. Following hybridization, the membrane is washed for 15 min at 25C. in 1 mM Tris (pH 8.0), 1% Sarkosyl, and four times for 15 min each at 25C. in 1 mM Tris (pH 8.0). To detect hybridization complexes, XOMAT-AR film (Eastman Kodak, Rochester N.Y.) is exposed to the membrane overnight at −70C., developed, and examined visually.

Polymer Coated Slide-Based Hybridization

Probe is heated to 65C. for five min, centrifuged five min at 9400 rpm in a 5415C. microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C. in 1×SSC, 0.1% SDS, and three times for 10 min each at 45C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

IX Complementary Molecules

Molecules complementary to the cDNA, from about 5 (PNA) to about 5000 bp (complement of a cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

X Protein Expression

Expression and purification of the protein are achieved using either a cell expression system or an insect cell expression system. The pUB6/V5-His vector system (Invitrogen, Carlsbad Calif.) is used to express tumor antigen in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autogiaphica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

XI Production of Antibodies

Tumor antigen is purified using polyacrylamide gel electrophoresis and used to immunize mice or rabbits. Antibodies are produced using the protocols below. Alternatively, the amino acid sequence of tumor antigen is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 43 1A peptide synthesizer (ABI) using Fmoc-chemistry and coupled to KLH (Sigma-Aldrich, St. Louis Mo.) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XII Purification of Naturally Occurring Protein Using Specific Antibodies

Naturally occurring or recombinant protein is purified by immunoaffinity chromatography using antibodies which specifically bind the protein. An immunoaffmity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIII Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIV Two-Hybrid Screen

A yeast two-hybrid system, MATCHMAKER LexA Two-Hybrid system (Clontech Laboratories, Palo Alto Calif.), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (−His), tryptophan (−Trp), and uracil (−Ura), and incubated at 30C. until the colonies have grown up and are counted. The colonies are pooled in a minimal volume of 1×TE (pH 7.5), replated on SD/−His/−Leu/−Trp/−Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (−Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/−Trp/−Ura liquid medium for 1 to 2 days at 30C. A sample of the culture is plated on SD/−Trp/−Ura media and incubated at 30C. until colonies appear. The sample is replica-plated on SD/−Trp/−Ura and SD/−His/−Trp/−Ura plates. Colonies that grow on SD containing histidine but not on media lacking histidine have lost the pLexA plasmid. Histidine-requiring colonies are grown on SD/Gal/Raf/X-Gal/−Trp/−Ura, and white colonies are isolated and propagated. The pB42AD-cDNA plasmid, which contains a cDNA encoding a protein that physically interacts with the protein, is isolated from the yeast cells and characterized.

XV Transcript Imaging

A transcript image was performed using the LIFESEQ GOLD database (Jun01release, Incyte Genomics). This process allowed assessment of the relative abundance of the expressed cDNAs in more than 1400 cDNA libraries. Criteria for transcript imaging can be selected from category, number of cDNAs per library, library description, disease indication, clinical relevance of sample, and the like.

All sequences and cDNA libraries in the LIFESEQ database have been categorized by system, organ/tissue and cell type. For each category, the number of libraries in which the sequence was expressed were counted and shown over the total number of libraries in that category. In some transcript images, all normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be disregarded or removed where clinical relevance is emphasized. Conversely, fetal tissue may be emphasized wherever elucidation of inherited disorders or differentiation of particular cells or organs from stem cells (such as nerves, heart or kidney) would be furthered by removing clinical samples from the analysis.

The transcript images for SEQ ID NOs: 1, 5, and 10 are shown below. The first column shows library name; the second column, the number of cDNAs sequenced in that library; the third column, the description of the library; the fourth column, absolute abundance of the transcript in the library; and the fifth column, percentage abundance of the transcript in the library.

Category: All (SEQ ID NO:1)

| Library* | cDNAs | Description of Prostate Tissue | Abundance | % Abund |
|---|---|---|---|---|
| CONDTUT01 | 1286 | peritoneum, neuroendocrine CA, 66F | 2 | 0.1555 |
| PENHTUE02 | 1846 | penis squamous cell CA, 64M, 5RP | 1 | 0.0542 |
| LUNGTUT09 | 3969 | lung squamous cell CA, 68M | 2 | 0.0504 |
| OVARTUM02 | 2932 | ovary papillary serous CA, 64F, WM/WN | 1 | 0.0341 |
| SPLNTUT02 | 3077 | spleen Hodgkin's, 45M | 1 | 0.0325 |
| COLITUT02 | 6656 | ileocecum, Burkitt lymphoma, 29F | 2 | 0.0300 |

*Cell line, fetal, pooled, subtracted and normalized libraries were not used in this analysis.

Differential expression of SEQ ID NO: 1 in neuroendocrine carcinoma of the peritoneum is 3-fold greater by percent abundance than expression in any other tissue of the digestive tract. No expression was found in cytologically normal tissue. When used in a cell or tissue specific diagnostic procedure and compared to established standards, SEQ ID NO: 1 is diagnostic for cancer, specifically neuroendocrine carcinoma, of the peritoneum.

Category: Exocrine (Breast)

| | | | | |
|---|---|---|---|---|
| Library* | cDNAs | Description of Bladder Tissue | Abundance | % Abund |

| | | | | |
|---|---|---|---|---|
| BRSTUNF01 | 1146 | breast tumor line T-47D, ductal CA, 54F | 1 | 0.0873 |
| BRSTTUT16 | 3724 | breast ductal CA, 43F, m/BRSTTMT01 | 2 | 0.0537 |
| BRSTTUT08 | 3928 | breast tumor, adenoCA, 45F, m/BRSTNOT09 | 2 | 0.0509 |
| BRSTUNT01 | 3130 | breast tumor line T47D, 54F | 1 | 0.0319 |
| BRSTNOT03 | 6777 | mw/BRSTTUT02 ductal adenoCA, 54F | 1 | 0.0148 |
| BRSTTUT13 | 7631 | breast adenoCA, 46F, m/BRSTNOT33 | 1 | 0.0131 |
| BRSTTUT03 | 10092 | breast lobular CA, 58F, m/BRSTNOT05 | 1 | 0.0099 |

*No libraries were excluded from this analysis

SEQ ID NO:5 is diagnostic of breast cancer as shown by its expression in breast tumor line T-47D and in these matched sets of cancerous and normal breast tissues. Expression was not found in cytological normal breast tissue removed from subjects during breast reduction surgery or any other breast library. When used with breast tissue, SEQ ID NO: 1 is diagnostic for breast cancer.

Category: Digestive Tract (Colon)

| Library | cDNAs | Description of Lung Tissue | Abundance | % Abund |
|---|---|---|---|---|
| COLNTUP12 | 2312 | colon adenoCA, M/F, pool, 3′ CGAP | 1 | 0.0433 |
| COLNTUP15 | 12065 | colon adenoCA, pool, NORM, 3′ CGAP | 5 | 0.0414 |
| COLNTUN03 | 2462 | colon adenoCA, M/F, pool, NORM | 1 | 0.0406 |
| COLNTUP17 | 7421 | colon adenoCA, 3′, CGAP | 2 | 0.0270 |
| COLITUT02 | 6656 | Burkitt lymphoma, 29F, m/COLANOT03 | 1 | 0.0150 |
| COLNTUP16 | 8499 | colon adenoCA, pool, NORM, 3′/5′ CGAP | 1 | 0.0118 |

Differential expression of SEQ ID NO: 10 was not found in libraries constructed from the tissues of subjects diagnosed with chronic ulcerative colitis (COLADIT05, COLANOT02, COLAUCT01, and COLDDIE01), benign familial polyposis (COLCDIT01, COLDNOT01, and COLTDIT04 ), ulcerative colitis (COLNDIP02, COLNNOT23, COLNUCT03, and COLSUCT01), or in cytologically normal tissue (COLNNON05, COLNNOP01, COLNNOP02, COLNNOT01, COLNNOT05, COLNNOT07, COLNNOT08, COLNNOT09, COLNNOT11, COLNNOT13, COLNNOT16, COLNNOT19, and COLNNOT22). When used in a cell or tissue specific diagnostic procedure and compared to established standards, SEQ ID NO: 1 is diagnostic for colon cancer.

In assays using established standards and patient samples, the cDNA, an mRNA, a protein or an antibody specifically binding the protein serves a clinically relevant diagnostic marker for cell cycle disorders.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A composition comprising a plurality of cDNAs having the nucleic acid sequences of SEQ ID NOs: 1-10 or the complements thereof.

2. A method for using a composition to detect gene expression in a sample containing nucleic acids, the method comprising:
a) hybridizing the composition of claim 1 to the nucleic acids under conditions for formation of one or more hybridization complexes; and
b) detecting hybridization complex formation, wherein complex formation indicates gene expression in the sample.

3. The method of claim 2 wherein the cDNAs of the composition are attached to a substrate.

4. The method of claim 7 wherein gene expression is compared to a standard and is indicative of a cell cycle disorder.

5. A method of using a composition to screen a plurality of molecules or compounds, the method comprising:
a) combining the composition of claim 1 with a plurality of molecules or compounds under conditions to allow specific binding; and
b) detecting specific binding, thereby identifying a molecule or compound that specifically binds a cDNA of the composition.

6. A cDNA comprising a nucleic acid sequence selected from SEQ ID NOs: 1, 2, 4-10 and a complement thereof.

7. A composition comprising the cDNA of claim 6 and a labeling moiety or a pharmaceutical carrier.

8. A method for using a cDNA to detect expression in a sample containing nucleic acids, the method comprising:
a) hybridizing the cDNA of claim 6 to the nucleic acids under conditions for formation of a more hybridization complex; and
b) detecting complex formation, wherein complex formation indicates expression in the sample.

9. The method of claim 8 wherein the cDNAs of the composition are attached to a substrate.

10. The method of claim 8 wherein expression is compared to a standard and is indicative of a cell cycle disorder.

11. A method of using a cDNA to screen a plurality of molecules or compounds to identify and purify a ligand, the method comprising:
a) combining the cDNA of claim 6 with a plurality of molecules or compounds under conditions to allow specific binding; and
b) recovering the bound cDNA;
c) dissociating the cDNA from the ligand thereby obtaining a purified ligand.

12. The method of claim 11 wherein the plurality of molecules or compounds is selected from DNA molecules, RNA molecules, peptide nucleic acids, transcription factors, enhancers, repressors, mimetics, and proteins.

13. An expression vector comprising a cDNA selected from SEQ ID NOs: 1, 2, and 4-10.

14. A host cell comprising the expression vector of claim 13.

15. A method for using a cDNA to produce a protein, the method comprising:
a) culturing the host cell of claim 14 under conditions for protein expression; and
b) recovering the protein from cell culture.

16. A purified protein or a portion thereof produced by the method of claim 15.

17. A composition comprising the protein produced by the method of claim 15 and a labeling moiety or a pharmaceutical carrier.

18. A method for using a protein to screen a plurality of molecules or compounds to identify and purify at least one ligand which specifically binds the protein, the method comprising:
a) combining the protein of claim 16 with the plurality of molecules or compounds under conditions to allow specific binding; and
b) recovering the bound protein;
c) dissociating the protein from the ligand thereby obtaining a purified ligand.

19. The method of claim 18 wherein the plurality of molecules is selected from DNA molecules, RNA molecules, peptide nucleic acids, mimetics, proteins, agonists, antagonists, and antibodies.

20. A method of using a protein to prepare and purify antibodies comprising:
a) immunizing an animal with the protein of claim 16 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.


SEQUENCE LISTING (10 sequences)

SEQ ID NO: 1 - DNA, 1970 bp, Homo sapiens
  misc_feature: Incyte ID No 040371.3
gggacttccagtaggaggcggcatgtttgaaaagtgatgacggttgacgtttgctgatttttgactttgcttgtagctgctccccgaactcgccgtcttcctgtcggcggccggcactgtaggtgagcgcgagaggacggaggaaggaagcctgcagacagacgccttctccatcccaaggcgcgggcaggtgccgggacgctgggcctggcggtgttttcgtcgtgctcagcggtgggaggaggcggaagaaaccagagcctgggagattaacaggaaacttccaagatggaaactttgtctttccccagatataatgtagctgagattgtgattcatattcgcaataagatcttaacaggagctgatggtaaaaacctcaccaagaatgatctttatccaaatccaaagcctgaagtcttgcacatgatctacatgagagccttacaaatagtatatggaattcgactggaacatttttacatgatgccagtgaactctgaagtcatgtatccacatttaatggaaggcttcttaccattcagcaatttagttactcatctggactcatttttgcctatctgccgggtgaatgactttgagactgctgatattctatgtccaaaagcaaaacggacaagtcggtttttaagtggcattatcaactttattcacttcagagaagcatgccgtgaaacgtatatggaatttctttggcaatataaatcctctgcggacaaaatgcaacagttaaacgccgcacaccaggaggcattaatgaaactggagagacttgattctgttccagttgaagagcaagaagagttcaagcagctttcagatggaattcaggagctacaacaatcactaaatcaggattttcatcaaaaaacgatagtgctgcaagagggaaattcccaaaagaagtcaaatatttcagagaaaaccaagcgtttgaatgaactaaaattgtcggtggtttctttgaaagaaatacaagagagtttgaaaacaaaaattgtggattctccagagaagttaaagaattataaagaaaaaatgaaagatacggtccagaagcttaaaaatgccagacaagaagtggtggagaaatatgaaatctatggagactcagttgactgcctgccttcatgtcagttggaagtgcagttatatcaaaagaaaatacaggacctttcagataatagggaaaaattagccagtatcttaaaggagagcctgaacttggaggaccaaattgagagtgatgagtcagaactgaagaaattgaagactgaagaaaattcgttcaaaagactgatgattgtgaagaaggaaaaacttgccacagcacaattcaaaataaataagaagcatgaagatgttaagcaatacaaacgcacagtaattgaggattgcaataaagttcaagaaaaaagaggtgctgtctatgaacgagtaaccacaattaatcaagaaatccaaaaaattaaacttggaattcaacaactaaaagatgctgctgaaagggagaaactgaagtcccaggaaatatttctaaacttgaaaactgctttggagaaataccacgacggtattgaaaaggcagcagaggactcctatgctaagatagatgagaagacagctgaactgaagaggaagatgttcaaaatgtcaacctgattaacaaaattacatgtctttttgtaaatggcttgccatcttttaattttctatttagaaagaaaagttgaagcgaatggaagtatcagaagtaccaaataatgttggcttcatcagtttttatacactctcataagtagttaataagatgaatttaatgtaggcttttattaatttataattaaaataacttgtgcagctattcatgtctctactctgccccttgttgtaaatagtttgagtaaaacaaaactagttacctttgaaatatatatatttttttctgt

SEQ ID NO: 2 - DNA, 1570 bp, Homo sapiens
  misc_feature: Incyte ID No 200394.1
cttaaaaagttgcagaaagaagaaaggaaagggaaagaaaagtgttcagaaatctttatatggggaaagagacattgcttctaagaagcccctcctcagtcctattcccgagctgcctgaagtccctgagatgacaccttccattccgagcatccgaagactgggttcaggttatttcagttcaaatggcaaactggaagaagtgaagactcctaaaaatccagtgaaaagaaaggatcttttgcgtcatgacccagatttgcatatgcatcaaggctatgataaatatgatgtctctgaattctgctctgatataaaaagttcctcatcgcttggcaatgctacttctgatgaagatccaaatacaaatataatgaacattaatgaaaataaaaatattccaaaagcaaaaaataagtcagaaagtgaaaatgaaccaaaagctggaactgacagtcctgtttcttgtgcttctataactgaagaacgtgtggcatcagatagtcccaaacctgctctgaccctgcagcagggtcaagaattttctgctggtggtcaaaatgcagaaaacctttgtcagttctttaaaatttcaccagatttaaacataaagtgtgaaagaaaggatgacttcttaggagctgcagaaggaaaactgcaatgcaatcgtttaatgcctaattcacaaaaagactgtcattgtttaggagatgtcttaattgaaaatacgaaagaatctaaaagccagagtgaggatttgggaagaaaacccatggaaagtagcagtgttgtgagttgcagagacaggaaagatagaagacgttccatgtgttattctgatggtcgaagtttacatttggaaaaaaatggaaatcacacaccatcctccagtgtgggcagctctgtagaaattagtttagaaaattctgaactgtttaaagatttgtctgatgccattgagcaaacctttcagaggagaaatagtgaaaccaaagtgcgacgtagcacgaggctacagaaggatttagaaaacgaaggtcttgtatggatttcacttccacttccttccacttcccaaaaagccaaaagaagaacaatatgtacatttgacagcagtggatttgaaagtatgtctcccataaaagaaactgtgtcctccagacaaaaaccgcagatggcacctcccgtctcagatccagaaaacagccagggccctgctgctggttcttccgatgaacctggtaagaggaggaagagcttttgtatatctacacttgcaaatactaaagccacttcccagttcaaaggctaccggagaagatcctctcttaatgggaagggagagagctctctgactgccttggaaaggattgaacataatggagaaagaaagcagtaattgacatttcctgcagagtctgtagcaagagggaaagtaaccatctatgctgaaatgatctgtctagttcccattctctgttcaacctcagtgtttcaaaagttcctaataaataaactcatttgagttgaacctacttttatgtagaaataaataagtttcttcatcatt

SEQ ID NO: 3 - DNA, 1324 bp, Homo sapiens
  misc_feature: Incyte ID No 201989.4
ctgttgtgcatccagaggtggaattggggcccggtaagtgatttgaataatttaataaataagttagagggctcagcaggcccagaacgagccattttgtcagctgcagcagtcattaactccgcagaggcctctggtccctcgccaggaagtttcttcactggaaactgggaagacagggtggtttgtaacttcgggagttgagccacgagctgttgtgcatccagaggtggaattggggcccggcattccctcctcgtcccgggctggcccttgccccccaccctgcaactcctggttgagatgggctcagccaagagcgtcccagtcacaccagcgcggcctccgccgcacaacaagcatctggctcgagtggcggacccccgttcacctagtgctggcatcctgcgcactcccatccaggtggagagctctccacagccaggcctaccagcaggggagcaactggagggtcttaaacatgcccaggactcagatccccgctctcctactcttggtattgcacggacacctatgaagaccagcagtggagaccccccaagcccactggtgaaacagctgagtgaagtatttgaaactgaagactctaaatcaaatcttcccccagagcctgttctgcccccagaggcacctttatcttctgaattggacttgcctctgggtacccagttatctgttgaggaacagatgccaccttggaaccagactgagttcccctccaaacaggtgttttccaaggaggaagcaagacagcccacagaaacccctgtggccagccagagctccgacaagccctcaagggaccctgagactcccagatcttcaggttctatgcgcaatagatggaaaccaaacagcagcaaggtactagggagatcccccctcaccatcctgcaggatgacaactcccctggcaccctgacactacgacagggtaagcggccttcacccctaagtgaaaatgttagtgaactaaaggaaggagccattcttggaactggacgacttctgaaaactggaggacgagcatgggagcaaggccaggaccatgacaaggaaaatcagcactttcccttggtggagagctaggccctgcatggccccagcaatgcagtcacccagggcctggtgatatctgtgtcctctcaccccttctttcccagggatactgaggaatggcttgttttcttagactcctcctcagctaccaaactgggactcacagctttattgggctttctttgtgtcttgtgtgtttcttttatattaaaggaagtaattttaaatgttactttaaaaaggt

SEQ ID NO: 4 - DNA, 1857 bp, Homo sapiens
  misc_feature: Incyte ID No 211475.1
ggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcataaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaggtgtctcagcattattttatacttc

SEQ ID NO: 5 - DNA, 2447 bp, Homo sapiens
  misc_feature: Incyte ID No 225657.4
ctccttcctcagcggcgggaagctggcggcagcggcggtggcggtggctgagcagaggacccggcgggcggcctcgcgggtcaggacacaatgtttgcacgaggactgaagaggaaatgtgttggccacgaggaagacgtggagggagccctggccggcttgaagacagtgtcctcatacagcctgcagcggcagtcgctcctggacatgtctctggtgaagttgcagctttgccacatgcttgtggagcccaacctgtgccgctcagtcctcattgccaacacggtccggcagatccaagaggagatgacgcaggatgggacgtggcgcacagtggcaccccaggctgcagagcgggcgccgctcgaccgcttggtctccacggagatcctgtgccgtgcagcgtgggggcaagagggggcacatcctgctcctggcttgggggacggccacacacagggtccagtttctgacctttgcccagtcacctcagcacaggcaccaaggcacctgcagagcagcgcctgggagatggatggccctcgagaaaacagaggaagctttcacaagtcacttgatcagatatttgaaacgctggagactaaaaaccccagctgcatggaagagctgttctcagacgtggacagcccctactacgacctggacacagtactgacaggcatgatggggggtgccaggccgggcccctgcgaagggctcgagggcttggctccggccaccccaggccctagctccagctgcaagtccgacctgggcgagctggaccacgtgatggagatcctggtggagacctgagcaggagccctgagtgctcacagccgcctctgacgcattgacacgtgagcactggctcccacggagggtgcgcctgccgccagcggcccagccttgctgccctgtctgctgattctgagaaatcccagaacagcccattaccagtggggctgcagccctaggcccgtcccactcacctcccccctgtggagggccaggcagaggctgttctggaaggcttcttgtcttctgacgtccccacagccctgggcccctcgtgtctctttgtgtcccccactgtagaggacggtgagccgcagctgcatcaacctccttttacctttagataggtgaatttttacaattcagttttacatgtttcgggcagtattttgtcttaagatatattttttaaactttttataccttatctctttagattttttcagctattttcttaaaagtatattttttctataaacatcctttgctgctacattagaacttttatagcctaaacaattgcagttggtgtgtttcatttttttaaggtttaaataagggttttttgttttgttttgttttttgcagtgagcatcactacagtctcagtcaacagtgtgaatgtatcatgttttactttaaatgtgtgtgtgatacttcttcattatgtcctgcgctgcagtgagacctgggtgaaaatcaggaaccgcacacagccacatcttcctagacctaagagtaaattatggaggattttatttatgtctatttatatgtaaatgtcattgaagacaaaggtcaaatatttgtctgtttgtagatcacaggcaccagttggtcttcagggacctcatagcccctcggtggtgccttctcaaggcagtgttcctggaggctcccgtcagggtcagcccatgcacctgccctgggtgaggaagtagcattgctgctggatgagaaacgcctgcgctgctctgttagactggtgctgaaacaaaaggttaaggctaggttgaagtctagaatgaaagaaatctgaatccatgtcattcataaccccttgatctgtagtgtcatgggtgctgccgcaggcagggagtgagctgggggtgcctgcagccttccactcctgccccgcctcaccccacatgctccctgtttctcatgctttctctaacttcctcaccccttaaccaaaaaggtgtgttttcttttgtgcatatagccattcttaaatatcagtgatgtaaacctcactttattaaaaaattatccagcaaacaaaatgggaatgtggtgttagttacgacccacggcctgaccctccagcaacctttctgcaggatcagttctgctgtattatctggtggtgctttctaaggtggggaaaggaattgcacttggctgcattaaatggacgctgggttacttttatttccccccccacagggttgcagagcaaattctttttacattgttcagcgcccggctggggttgggggtgtccacgacctctgacagcccccgatgtcgaaagttaatcctcatggaccctagtttaaagggtatgtattttataggaataaatctaaagcactattttgtttctgtatagcatttttatcttttagaaacatcatttgttcagc

SEQ ID NO: 6 - DNA, 2482 bp, Homo sapiens
  misc_feature: Incyte ID No 350770.3
gcgagtggccttcccggttggcgcgcgcccggggcggcggcgctggaggagctcgagacggagcctagttatgtctgggaggcgaacgcggtccggaggagccgctcagcgctccgggccaagggccccatctcctactaagcctctgcggaggtcccagcggaaatcaggctctgaactcccgagcatcctccctgaaatctggccgaagacacccagtgcggctgcagtcagaaagcccatcgtcttaaagaggatcgtggcccatgctgtagaggtcccagctgtccaatcacctcgcaggagccctaggatttcctttttcttggagaaagaaaacgagccccctggcagggagcttactaaggaggaccttttcaagacacacagcgtccctgccacccccaccagcactcctgtgccgaaccctgaggccgagtccagctccaaggaaggagagctggacgccagagacttggaaatgtctaagaaagtcaggcgttcctacagccggctggagacccctggggctctgcctctacctccaccccaggccgccggtcctgctttggcttcgaggggctgctgggggcagaagacttgtccggagtctcgccagtggtgtgctccaaactcaccgaggtccccagggtttgtgcaaagccctgggccccagacatgactctccctggaatctccccaccacccgagaaacagaaacgtaagaagaagaaaatgccagagatcttgaaaacggagctggatgagtgggctgcggccatgaatgccgagtttgaagctgctgagcagtttgatctcctggttgaatgagatgcagtggggggtgcacctggccagactctccctcctgtcctgtacatagccacctccctgtggagaggacacttagggtcccctcccctggtcttgttacctgtgtgtgtgctggtgctgcgcatgaggactgtctgcctttgagggcttgggcagcagcggcagccatcttggttttaggaaatggggccgcctggcccagccactcactggtgtcctgctcttgtcgtcctgtccttcctatctccccaaagtaccatagccagtttccagatgggccacagactggggaggagaatcagtggcccagccagaagttaaagggctgagggttgaggtgagaggcacctctgctcttgttgggaggggtggctgcttggaaataggcccaggggctctgccagcctcggcctctccctcctgagttgccttctgttggtggctttcttcttgaacccacctgtgtaaagaggttttcagttccgtgggtttcccctttgattctgtaaatagtcccagagagaattcgtgggctgagggcaattctgtcttggaggaagaagctggacattcagcctgtggagtctgagttttgaaggatgtagggagccttagttgggtctcagaccataagtgtgtactacacagaagctgtgttttctagttctggtctgctgttgagatgtttggtaaatgccaggttgatagggcgctggctgcttggagcaaagggtgcatttcagggtgtggccaccaggtgctgtgagtttctgtggctcatggcctctgggctggtcccttgcacagggcccacgctggagtcttaccactctgctgcaggggtggaaggtggcccctcttgtcacccatacccatttcttacaaaataagttacaccgagtctacttggccctagaagagaaagttgaagagtcccagacctactagcattttgcaactatgcttgtaaagtcctcggaaagtttcctcgcgtaccagacagcggcgggggctgatagcaattttagtttttggcctccctatcctctcacatgagaacactgcctggatgcatctcatgatctctggagaatttccccatctttctcttctttccatcgtgtggattcaatagtgtggatttgaaggctgccctgcccccgactctcctgccgcacccctggccattgtaccttttgatgtttagaagttcgtggaagtagacgctgaggtgtgcagaggagctggtggataacagagaatgccagggaagatgagtgctgggtcagggtacttggatgaaacggtgcaggccaggcgggccctaataaaaccctctgccaggtctgggagtcccaggccatctgctcaacgctctgtggtttgtcagacctgcaagcaagccccctgctggggaagcctaggtgtccttgagctgaaccgcactgaagaactcttgtcctcactggctgatgcagcagaactcttgggaaatgtcttagtcctgcagaatcaggagtcaccagatgatgcagagttgagatcatcattgcaaagttctctgttcctgaggaactaaatttaaggaaaaaatgggattttgttttagagttggaaaaaaaacctgattaaagagtttctgcctgtt

SEQ ID NO: 7 - DNA, 2405 bp, Homo sapiens
  misc_feature: Incyte ID No 407614.1
aagggacttctcccgcaccccactctgtcccaggacatagggcagggggcctcactgccttgttggtctccaccttgttcctacctctgcaggcctctttgctctcccctcttgcctcaggaaacccggtggcacctgtggctccaggtgactgtcttgaacagagcgggcttcttcatggctgcgttgttgctgagtttgaactgctcctccctggcctgcgtgactgaatcacagctttggtccctgtcttgcaggggctgaggtgtcaggaggggacttctggcccaccttgccttcagccctggagtgggcagagagtattgtggggaggcatggccagtgggactagtgttccctccatctggccacagcttttgggagatggggtgggcaggggtggtcctggctggcattgcctgagccggccagtgatgaagtggggagcttgcccttgacaggtgggggctggctggggccttaatgtgaaaagacagtggcaggcagctggagtagagcgagcccagcagccctaaaaggctgccttcatggccatctagccccagttcagggcagcatccatagcccacaagccagcgtgggtggggcgggggtggtcccacagctgggttccacctgaagagcctccgtgcctcggagcaggagaggcaggctatggctgccaccctccctcctgcctgtgtcccagtgagaactgacctgagtccccttccaaacccagacccacctcctgccccaggcccactgaagcatgttccatttctaaaaagcccagagttcagtgtgtcccaaggaaaacccaaagtggaggtgctcaggtccaggggagtccagtgggcaggacccttggcaggcaagcccctcccttcactcccaggacctaccttctgctagtaaaggactaggcttcattctaattatggcccacagactgccccggagacctggaggacagcagtgctggcacttgggtgtccatgggcccgtctgccggctctgcctgtgctgcaagtgttggccgtgggtccagccaacaactccctacgtcctgtgtggggccctgcccaagtggatgaggcattccttgaggagtatcattttccctgacaatccccatcacctttaggggttccctgcttggctcctttccagctgaaaaactagacctgtgccattggggaagctggacaaagtctagggggcccgcctggtagagggtcccgggaagctggatctgtcagcctcggccctgaggcccctgttaactcaagactgtgagctgcctctaggtggtcacgtctgggagctagcttgtatggcttctgaccagtatcaggatttctgttctgagagcagcgtgggcagcaaggcagggcagcccagaggtggcagcggcaggcaatctggtcactaggtctttgtgatgccaaaaataaaagagggtggggtgggtgctttctgttcctctgattggatggagtccgccagcaggcatggggctacattccagtgcctgactatagggaggcactcctgattccatggagcagcccggactttgagaatgggctctggtttgcggggggcaggcgtaccagactgcaagaccccccagtacctcaccgtgccaaataggaagaggtggccttggtgtagccaaatggatctttttaacagtgtgcctttggggagggacccatgtccatggcttcgttgagggccatccatatgccagctgggggccagcccacagtggcccatgttggctgcagcaggaatggtgcccacctcggcgaattgaagggctaagagtcccagatagctagggccagagctggaagcagacagtaaggggaagagctgctcccacaggagagggagagattccagctcactgcgcagcctgggaggaggcgtggatcctggcacgctgagcctcaggcaccagcctccctgtgctcgacagcaaagtcttgactccttcctgctgagcactgtgctaccttcactgctccaaagccagactaacagctctccaagcccttggggtgactcggcttccaggagctgttggagaaatgaggatgtctgtccctgtctgcctgggcaggccagattcctccccagcagccgggtctctccagaccctgattcggtgcctttctgtttaccagctacttcaatcccaaagtttgaatctgcagataccttactcccagccactttgccttcttactgtgttgtgtgtttttcctggtgcttcaagagcgtgtgcagggcaagtgccgtcactgggaactgcaccagatgctcagacttggttgtcttatgtttaccaataaataaaagtagactttttctatttttatttgctgctaaaaaaa

SEQ ID NO: 8 - DNA, 2159 bp, Homo sapiens
  misc_feature: Incyte ID No 475113.7
agagtcccgccagccctcagagaattctgtgactgattccaactccgattcagaagatgaaagtggaatgaattttttggagaaaagggctttaaatataaagcaaaacaaagcaatgcttgcaaaactcatgtctgaattagaaagcttccctggctcgttccgtggaagacatcccctcccaggctccgactcacaatcaaggagaccgcgaaggcgtacattcccgggtgttgcttccaggagaaaccctgaacggagagctcgtcctcttaccaggtcaaggtcccggatcctcgggtcccttgacgctctacccatnnnnnnnnnnnnnnnnnnnnnnnnntacatgttggtgagaaagaggaagaccgtggatggctacatgaatgaagatgacctgcccagaagccgtcgctccagatcatccgtgacccttccgcatataattcgcccagtggaagaaattacagaggaggagttggagaacgtctgcagcaattctcgagagaagatatataaccgttcactgggctctacttgtcatcaatgccgtcagaagactattgataccaaaacaaactgcagaaacccagactgctggggcgttcgaggccagttctgtggcccctgccttcgaaaccgttatggtgaagaggtcagggatgctctgctggatccgaactggcattgcccgccttgtcgaggaatctgcaactgcagtttctgccggcagcgagatggacggtgtgcgactggggtccttgtgtatttagccaaatatcatggctttgggaatgtgcatgcctacttgaaaagcctgaaacaggaatttgaaatgcaagcataatatctggaaaatttgctgcctgccttctacttctcaaatctttcttgtaaaagtttccaattttttcactgaaacctgagttaaaaatcttgatgatcagcctgtttcataagaaactccaatcaagttaatcttagcagacatgtgtttctggagcatcacagaaggtatattgctagttacactttgccctcctgcagtttcttctctgctcccaacccccatctcatagcatccccctctatttccaatgctcctctccaaccgcttagtttctgaatttcttttaaattacagttttatgaaagcatattttatttacttggtgttgaaatagccctcataaaacctaagcacttggaaacacaataatagtattaactaactagatctattgaatttcagagaagagccttctaacttgtttacacaaaaacgagtatgatttagcattcatactagttgaaatttttaatagaatcaaggcacaaaagtcttaaaaccatgtggaaaaattaggtaattattgcagattgatgtctctcaatcccatgtattgcgcttatgttacaagttgttgtcacagttgagacttaatttctcctaatttcttctgcccgaagggtaagtggtgccgtccagcttacacaatcataattcaaaggttggtgggcaatgtaatacttaattaaaataatgatggaagagctatctggagattatgagtaagctgatttgaattttcagtataaaactttagtataattgtagtttgcaaagtttatttcagttcacatgtaaggtattgcaaataaattcttggacaattttgtatggaaacttgatattaaaaactagtctgtggttctttgcagtttcttgtaaatttataaaccaggcacaaggttcaagtttagattttaagcacttttataacaatgataagtgcctttttggagatgtaacttttagcagtttgttaacctgacatctctgccagtctagtttctgggcaggtttcctgtgtcagtattccccctcctctttgcattaatcaaggtatttggtagaggtggaatctaagtgtttgtatgtccaatttacttgcatatgtaaaccattgctgtgccattcaatgtttgatgcataattggaccttgaatcgataagtgtaaatacagcttttgatctgtaatgcttttatacaaaagtttattttaataataaaatgtttgttctaacttgtctgcttttttaaaaataatcttactgtacttaattctaattttttcctcatatttaaataaaa

SEQ ID NO: 9 - DNA, 535 bp, Homo sapiens
  misc_feature: Incyte ID No 898622.1
cccaaagtgctgagattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcaaaaaaaa

SEQ ID NO: 10 - DNA, 2373 bp, Homo sapiens
  misc_feature: Incyte ID No 978267.1
ggttgactgtagagccgctctctctcactggcacagcgaggttttgctcagcccttgtctcgggaccgcaggtacgtgtctggcgacttcttcgggtggtccccgtccgccctcctcgtccctacccagtttcttgcttccctgccccatctccgccgctccccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagagggaagaaggagaaggaaaatttacgtaagaatcttcaaactgcaagagtcaaaaggtgtcctccatccaagaagagaactcagtccatacaaggcaaaggaaaagggaaaaggtcaagccgtgctaacactgttaccccagccgtgggccgattggaggtgtccatggtcaaaccaactccaggcctgacacccaggtttgactcaagggtcttcaagaccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccagctgaatcctccctgctctctgagctgctgccttttgcctcctgcaactcaacatcctcttcaccctgccctgcctgcagttgagggggcgaagaagaaccctgtgttctcaggaagactgcctccaccaccgctacccagagaacctctgcatctggcatttctgctctctatgcttgagaccgggaggtttaggctcagataagtgagctctgggccatgagagggtaggtccagaaggtggggggaactgtacagatcagcagagcaggacagttggcagcagtgacctcagtagggaacatgtccgtctaccctctcgcactcatgacacctccccctaccagcctctctctctctcacctcctctgtgggaggtggtcagtgggacttagggatctttcacctgctgtgcccagtagttctgaagtctgcttgtggagcagtgttttatgtttatccctgtttactgaagaccaaatactggtttggagacaacttccatgtcttgctcttctacctccctagttagtggaaatttggataagggaactgtagggcccagattctggaggttttatgtcattggccacagaataactgtctctaagctatccatggtccagtggtccctgccaagtctgtagacttcagagagcacttctctcttatggggttcatgggaacaggggcgggtgtgacttgcttggtggcctcattccatgtgtgcctgtgcctggggcatggactttgttaagcagagtcagcagtgaggtcctcattctccagccagcctctctgccctggagaatcatgtgctatgttctaagaatttgagaactagagtcctcatccccaggcttgaaggcacatggctttctcatgtagggctctctgtggtatttgttattattttgcaacaagaccattttagtaaaacagtcctgttcaagttgtattcttttaagttcttttattctcctttccctgagatttttgtatatattgttctgagtaatggtatctttgagctgattgttctaatcagagctggtacctactttcaataaattctggttttgtgttttcttttgt